(19) United States
(12) Patent Application Publication
(10) Pub. No.: US 2010/0016267 A1
(43) Pub. Date: Jan. 21, 2010

(54) Pharmaceutical compositions for administraton to a sinus

(76) Inventors: Felix Theeuwes, Los Altos Hills, CA (US); Arthur J. Tipton, Birmingham, AL (US)

Correspondence Address: DURECT CORPORATION;THOMAS P. MCCRACKEN, 2 RESULTS WAY, CUPERTINO, CA, 95014, US

(21) Appl. No.: 10/592,886

(22) PCT Filed: Mar. 15, 2005
(86) PCT No.: PCT/US05/08743

§ 371 Date: Oct. 3, 2008

Related U.S. Application Data
(60) Provisional application No. 60/553,190, filed on Mar. 15, 2004.

(51) Int. Cl. A61K 31/58; A61P 27/00

(52) U.S. Cl. 514/172

(57) ABSTRACT

Pharmaceutical compositions for delivering a drug to a sinus in a subject are provided. The compositions are formed by the combination of a carrier material and a drug and are specially adapted for delivery to a sinus. The compositions have no fixed shape, and the carrier material serves to increase the viscosity or specific volume of the composition after introduction of the composition into the sinus. Also provided are methods for delivering a drug to a subject. The methods entail administering a pharmaceutical composition according to the present invention directly to a sinus. The drug is then released from the composition to the sinus. The pharmaceutical compositions can be administered to a sinus in liquid form, for example, as a suspension or solution using standard techniques. Also provided is the use of a carrier material in the manufacture of a drug delivery composition. The drug delivery composition is for delivering a drug to a sinus in a subject. The composition comprises the carrier material combined with the drug to be delivered, and has no fixed shape. After the composition is introduced into a sinus, the carrier material serves to increase the viscosity or specific volume of the composition.

### FIELD OF THE INVENTION

The invention relates to pharmaceutical compositions in general, and more specifically to compositions suitable for administration directly to a sinus in a subject.

### BACKGROUND OF THE INVENTION

The paranasal sinuses are air-filled cavities within the facial skeleton. Each paranasal sinus is contiguous with a nasal cavity and drains into the nose through a sinus ostium. Although other factors may be involved, the development of sinusitis (inflammation of the mucosal lining of the sinuses) is most often attributed to blockage of one or more of these sinus ostia, followed by mucostasis and microbial overgrowth in the sinus cavity. Ostial blockage may stem from predisposing anatomical factors, or inflammation and edema of the mucous lining in the area of the ostia, arising from such etiologies as viral or bacterial upper respiratory infection or chronic allergic processes.

Sinusitis is one of the most commonly encountered health care problems in many areas of the world. It can have significant adverse effect on quality of life, particularly when present as a chronic disease. Traditionally, sinusitis has been medically managed by the oral administration of antibiotics and steroids. However, penetration of these systemically delivered agents into the sinus mucosa is limited due to poor blood flow to the sinuses. Therapeutic agents contained in aqueous solutions, creams, or gels, for topical application in the nose have also been formulated, but usually never travel far enough into the nose to reach the sinuses, are blocked from entering the sinuses due to obstructed ostia, or have such short contact with the sinus mucosa that absorption of the agent is low. For similar reasons, nasally inhaled steroid and anti-infective aerosols that have been developed to treat sinusitis are equally ineffective.

The delivery of ampicillin from a poly(lactic-co-glycolic)acid (PLGA) film to increase residence time of the antibiotic in rabbit sinuses has been investigated for the treatment of sinusitis (Min et al. Mucociliary Activity and Histopathology of Sinus Mucosa in Experimental Maxillary Sinusitis: A Comparison of Systemic Administration of Antibiotic and Antibiotic Delivery by Polylactic Acid Polymer. Laryngoscope 105:835-342 (1995) and Min et al. Application of Polylactic Acid Polymer in the Treatment of Acute Maxillary Sinusitis in Rabbits. Acta Otolaryngol 115:548-552 (1995)). Although clinical signs of sinusitis improved, the procedure for placing the film in the sinus required that a hole be drilled through the anterior wall of the maxillary sinus.

An attempt to lengthen the time of delivery of drugs to the paranasal sinuses is described in Piskunov et al., The Prolongation of Drug Action in the Treatment of Diseases of the Nose and Paranasal Sinuses, Phinology 31:33-36 (1993). A water-swellable composition containing cellulose polymers and polyvinyl alcohol is described. However, the formulations described contain water added to the formulation, and are thus in gel form prior to administration, requiring a larger bore needle to deliver the composition.

Attempts to treat chronic rhinosinusitis without surgical intervention (which can increase inflammation) are described in Lavigne et al., Intrasinus Administration of Topical Budesonide to Allergic Patients with Chronic Rhinosinusitis Following Surgery, Laryngoscope, 112: May 2002. Topical corticosteroids are administered directly into the maxillary sinus. However, the process described uses a fairly large bore “maxillary antrum sinusotomy tube,” rather than a small bore needle. The article does not describe any attempts to formulate the corticosteroid in a controlled release form, or in a form that would allow less intrusive and less traumatic administration to the sinus cavity.

Other techniques for intrasinus administration of biologically active materials are known in the art. However, they generally include the introduction of implants, containing biodegradable polymer and a biologically active material, into the maxillary sinus cavity. These materials and techniques suffer from several disadvantages.

Because of their pre-formed solid nature, implantation generally requires use of a large bore needle, increasing pain and discomfort resulting from administration. Moreover, although a large bore needle is required for implantation, the implants themselves may be quite small relative to the size of the sinus cavity. Delivery of active compounds in the implant thus may not show reliable mass diffusion and biological transfer to move the compounds from the implant to the surfaces of the sinus cavity. In addition, implants do not conform to the cavity and may result in erratic delivery profiles. As a result, it is difficult to obtain consistent distribution of active materials to all surfaces in the sinus cavity.

Retention and clearance of the implanted polymeric solids may also be problematic; the mucociliary lining of the sinuses tend to transport materials out of the sinus. Implanted polymeric solids (e.g., films) can thus potentially obstruct the sinus cavity and aggravate the initial problem or exit the sinus cavity into which they were implanted and travel to other parts of the patient's body. Attempts have been made to adhere the implanted solids to the sinus lining, but generally require the presence of a compound specifically designed to function as a bio-adhesive.

### SUMMARY OF THE INVENTION

Drug delivery compositions adapted for administration to a sinus in a subject and suitable for delivering a drug of interest are provided. It is thus an object of the present invention to provide for the use of a carrier material for the manufacture of a drug delivery composition for delivering a drug to a subject, where the composition includes the carrier material combined with a drug of interest and is adapted specifically for administration directly into a sinus in the subject.

It is more particularly an object of the present invention to provide for the use of a carrier material for the manufacture of a drug delivery composition for delivering a drug to a subject, where the composition includes the carrier material combined with a drug of interest and is adapted specifically for administration directly into a sinus in the subject. The composition has no fixed initial shape, and the carrier material serves to increase the viscosity or specific volume of the composition after it is introduced into the sinus. The compositions manufactured in accordance with the invention can thus provide for safe and effective local and/or systemic intrasinus administration of drugs, and such administration can be accomplished with decreased levels of discomfort and invasiveness.

In certain aspects of the invention, the carrier material is used to manufacture a composition that forms a gel, semi-solid, foam or high viscosity liquid after introduction into the sinus. In one particular aspect, the carrier material is used to manufacture a composition that forms a drug delivery depot in situ in the sinus. In certain instances, the increase in the viscosity or specific volume of the composition after administration into a sinus is suitable to fill at least a portion of a targeted sinus space, and in other cases it will fill the entire sinus space. In this manner, the composition both forms a drug delivery depot in situ and maintains good contact with the tissue walls of the sinus space, or will coat the inner surfaces of the sinus space. Contact with the sinus tissue provides a mechanism for transfer of the drug of interest to the subject, providing for effective local and/or systemic intrasinus administration.

In certain other aspects of the invention, the carrier material is used to manufacture a composition that is adapted to at least partially clear from the sinus after delivering an effective amount of the drug. Such clearance is beneficial since the composition does not need to be retrieved from the sinus after drug delivery has been completed. Clearance of the composition can be due to the composition being metabolized, or cleared, or both, by processes occurring naturally in the subject including any biodegradation process. Accordingly, certain compositions as manufactured herein are wholly or at least partially biodegradable. In addition, the carrier material can preferably confer controlled release properties upon the composition, thereby providing beneficial drug release profile characteristics from the composition after administration to the sinus. In still further aspects of the invention, the carrier material comprises a high viscosity liquid carrier material (HVLCM), for example, a sucrose acetate isobutyrate.

In still other aspects of the invention, the composition is manufactured to include a further ingredient, or a combination of further ingredients (e.g., biodegradable polymers, solvents, diluents, stabilizers, viscosity modifying agents, etc. disclosed below) that allow the composition to exist in a liquid form prior to administration, but upon introduction into the sinus the composition can either form or revert back to a gel, or attain a semi-solid or solid form. In certain compositions, the additional ingredient can be a solvent or other viscosity modifying agent that, due to diffusion or evaporation (or both), will be transported away from the composition and thus provide for an increase in the viscosity of the remaining composition. In other aspects of the invention, the composition is manufactured to include a mucoadhesive agent suitable to help retain the composition in the sinus. The ability to provide the composition in liquid form allows for administration of the composition using standard injection (e.g., needle and syringe) techniques, or by spraying (e.g., by atomization or use of a propellant).

In the practice of the invention, the carrier material can be used to manufacture a composition that includes any drug or active agent of interest, including for example, an antifungal agent, anti-inflammatory agent, anti-infective agent, or any combination thereof. In certain compositions, the drug is mometasone.

It is another object of the present invention to provide a method for delivering a drug to a subject. The method entails administering a drug delivery composition directly to a sinus in the subject, where the composition includes a carrier material combined with the drug of interest, and the composition has no fixed shape. The drug is released from the composition to the sinus after administration. The carrier material serves to increase the viscosity or specific volume of the composition after it is introduced into the sinus. The methods of the invention can thus provide for safe and effective local and/or systemic intrasinus administration of drugs, and such administration can be accomplished with decreased levels of discomfort and invasiveness.

In certain aspects of the invention, the composition used in the method forms a gel, semi-solid, foam or high viscosity liquid after introduction into the sinus. In one particular aspect, the composition can form a drug delivery depot in situ in the sinus. In certain instances, the increase in the viscosity or specific volume of the composition after administration into a sinus is suitable to fill at least a portion of a targeted sinus space, and in other cases it will fill the entire sinus space. In this manner, the composition both forms a drug delivery depot in situ and maintains good contact with the tissue walls of the sinus space, or will coat the inner surfaces of the sinus space. Contact with the sinus tissue provides a mechanism for transfer of the drug of interest to the subject, providing for effective local and/or systemic intrasinus administration.

In certain other aspects of the invention, the composition that is used in the method is adapted to at least partially clear from the sinus after delivering an effective amount of the drug. Such clearance is beneficial since the composition does not need to be retrieved from the sinus after drug delivery has been completed. Clearance of the composition can be due to the composition being metabolized, or cleared, or both, such as by processes occurring naturally in the subject including any biodegradation process. Accordingly, certain compositions used in the instant methods are wholly or at least partially biodegradable. In addition, the carrier material can preferably confer controlled release properties upon the composition, thereby providing beneficial drug release profile characteristics from the composition after administration to the sinus. In yet further aspects of the invention, the carrier material comprises a high viscosity liquid carrier material (HVLCM), for example, a sucrose acetate isobutyrate. In still other aspects of the invention, the composition used in the practice of the method includes a further ingredient, or a combination of further ingredients (e.g., biodegradable polymers, solvents, diluents, stabilizers, viscosity modifying agents, etc. disclosed below) that allow the composition to exist in a liquid form prior to administration, but upon introduction into the sinus, the composition can either form, or revert back to a gel, semi-solid or solid form. In certain compositions, the additional ingredient can be a solvent or other viscosity modifying agent that, due to diffusion or evaporation (or both), will be transported away from the composition and thus provide for an increase in the viscosity of the remaining composition. In other aspects of the invention, the composition includes a mucoadhesive agent suitable to help retain the composition in the sinus after administration. The ability to provide the composition in liquid form allows for administration of the composition using standard injection (e.g., needle and syringe) techniques, or by spraying (e.g., by atomization or use of a propellant).

In the practice of the invention, the methods can be used to deliver any drug or active agent of interest, including for example, an antifungal agent, anti-inflammatory agent, anti-infective agent, or any combination thereof. In certain methods, the drug that is delivered is mometasone.

It is an advantage of the present invention that the compositions can serve as a platform for delivery of a wide variety of drugs to the tissues of the sinus. In addition, in certain compositions the bulk of the delivered composition does not need to be retrieved from the sinus after the drug delivery event since the composition can be metabolized, cleared, or both, by naturally occurring processes such as biodegradation.

It is a further advantage of the invention that the compositions can be provided in liquid form, allowing it to be readily and easily administered to a target sinus. The compositions can further take the form of the targeted sinus area, or easily be distributed throughout the sinus, and/or can readily cross the internal surfaces of the sinus. In those compositions that increase in viscosity after administration, the high viscosity allows the compositions to remain within the sinus, and makes it difficult for a mucociliary layer to transport the composition away. The compositions can provide controlled release of drug in the sinus, can be more easily administered, e.g. using a small-bore needle, and can be retained for a longer duration in the sinus without the need for specific adhesive materials.

In addition, various physical properties of the compositions can be readily provided by the inclusion of various compatible ingredients. For example, additional ingredients can be included to increase the ability of the compositions to fill a sinus cavity or a portion thereof. Other ingredients can be added to increase the muco-adhesive nature of the compositions, providing the ability to avoid clearance by cilia and other transport mechanisms. A still further advantage is that the compositions of the present invention can be modified by including ingredients that alter the drug release profile characteristics of the composition, allowing for the delivery of a wide variety of different drugs. Any problems associated with systemic administration of drugs because of limited blood flow to the intrasinus membranes can be reduced or eliminated by the inclusion of vasoactive (e.g., vasodilating) materials in the composition, which can be used to increase blood flow to the membranes.

Finally, in those compositions that form a foam after introduction to the sinus, there is a further advantage in that the composition (and thus the drug) can be well distributed in the targeted sinus and contact all or most of the sinus tissue surfaces without requiring the bulk of a solid material.

These and other objects, aspects and advantages of the present invention will readily occur to the skilled practitioner upon reading the instant disclosure and specification.

### DETAILED DESCRIPTION OF SPECIFIC EMBODIMENTS

Before describing the present invention in detail, it is to be understood that this invention is not limited to particularly exemplified carrier materials or delivery parameters as such may, of course, vary. It is also to be understood that the terminology used herein is for the purpose of describing particular embodiments of the invention only, and is not intended to be limiting.

All publications, patents and patent applications cited herein, whether supra or infra, are hereby incorporated by reference in their entirety.

It must be noted that, as used in this specification and the appended claims, the singular forms “a,” “an” and “the” include plural referents unless the content clearly dictates otherwise. Thus, for example, reference to “a carrier” includes a mixture of two or more such carriers, reference to “a solvent” includes a mixture of two or more such solvents, reference to “a drug” includes two or more drugs, reference to “an excipient” includes mixtures of two or more such excipients, and the like.

### DEFINITIONS

For purposes of this description and the attached claims, we use the following terms as defined in this section.

As used herein, the term “sinus” refers to all sinuses generally, that is, a cavity, channel or hollow in bone or other tissue, and in particular the term includes all nasal or paranasal sinuses (cavities in the bones of the face that are continuous or connected with the nasal cavity), thus encompassing the maxillary, ethmoidal, frontal, and sphenoidal sinuses. The term further refers to the cavities, channels and hollows per se, as well as all surrounding cells and tissue.

The term “subject,” as used herein, refers to any vertebrate. The term is used interchangeably with “individual” and “patient” and thus broadly refers to any vertebrate animal that is to be treated or otherwise contacted with the compositions of the present invention, such as birds, fish and mammals including humans. In certain cases, the compositions of the present invention are suitable for use in veterinary practice and animal husbandry, e.g., the treatment and/or care of birds and mammals. In certain other cases, the compositions are particularly suited for use with companion animals such as dogs or cats, and additionally may be used with horses. In preferred methods, the term “subject” intends a human subject. Furthermore, the term “subject” does not denote a particular age, and the compositions of the present invention are thus suited for use with subjects of any age, such as infant, adolescent, adult and senior aged subjects.

As used herein, the terms “treat,” “treating,” or “treatment” refer to the resolution, care, reduction, or prevention of injury or disease condition, and thus include prophylactic, therapeutic and palliative techniques.

The term “composition”, used interchangeably herein with “formulation”, is used in its broadest sense, that is, to refer to a product obtained by mixing or otherwise combining a drug with a carrier and, optionally, other elements or ingredients to provide a pharmaceutically suitable product.

As used herein, the terms “drug” and “therapeutic agent” are used interchangeably and refer to any molecule or substance used internally or externally as a pharmacological material or medicine for the treatment, amelioration, cure, or prevention of a disease, condition or disorder.

As used herein, the terms “therapeutic amount,” “therapeutic dose,” or “effective amount” refer to concentrations or amounts of therapeutic agents or drugs present in a composition that are appropriate to safely treat, ameliorate or prevent an injury, disease or condition in a subject.

A “high viscosity liquid carrier material” or “HVLCM” refers to a non-polymeric, non-water soluble liquid material having a viscosity of at least about 5,000 cP at 37° C., wherein the liquid material does not crystallize neat under ambient or physiological conditions. A HVLCM may be carbohydrate-based, and may include one or more cyclic carbohydrates chemically combined with one or more carboxylic acids, such as sucrose acetate sucrose acetate isobutyrate (SAIB) or some other ester of a sugar alcohol moiety with one or more alkanoic acid moieties. A HVLCM may alternatively be a non-polymeric ester or mixed ester of one or more carboxylic acids, having a viscosity of at least about 5,000 cP at 37° C., and that does not crystallize neat under ambient or physiological conditions, wherein when the ester contains an alcohol moiety (e.g., glycerol), the ester may for example comprise from about 2 to about 20 hydroxy acid moieties. Various HVLCMs used in the practice of the present invention are described in U.S. Pat. Nos. 5,747,058; 5,968,542; and 6,413,536.

The term “gel” as used herein refers to a colloidal dispersion where the dispersed phase and the continuous phase form a viscous, jelly-like material that is not flowable except under high applied shear stress (e.g., a shear stress typically higher than about 10 dynes/cm²).

The term “semi-solid” as used herein refers to viscous, jelly-like materials that are not colloidal dispersions, and that are not flowable except under very high applied shear stress.

The term “foam” as used herein refers to a dispersion of a gas in a liquid, solid, or semisolid or gel. The terms “foaming agent” or “blowing agent” as used herein refer to gaseous materials formed in, or introduced into a liquid, solid, semisolid, or gel in order to provide a foam.

### Drug Delivery Compositions

Drug delivery compositions that are specially adapted for direct administration to a sinus in a subject and then delivering a drug of interest to the sinus are provided herein. More particularly, the present invention provides for the use of a carrier material for the manufacture of a drug delivery composition for delivering a drug to a subject, wherein the composition includes the carrier material combined with a drug of interest, and is adapted specifically for administration directly into a sinus in the subject. Methods of using such compositions are also provided herein. The instant compositions allow for effective local and/or systemic intrasinus administration of any drug of interest.

The drug delivery compositions are characterized in that they either increase in viscosity or increase in specific volume upon or after introduction into the sinus of a subject. In this manner, the compositions will likely lead to a much higher level of patient and physician acceptance since the compositions can be prepared and administered in liquid form (e.g., suspension or solution) and will then form a high viscosity liquid, gel, semisolid, solid or a foam in situ after introduction into the sinus.

It is an advantage of the present invention in that the compositions can be provided as a liquid having a lower viscosity than it has after introduction into the sinus. More particularly, the compositions in their pre-introduction form can be a liquid of sufficiently low viscosity (e.g., less than about 50,000 cP, less than about 5,000 cP, and more particularly less than about 1,000 to 2,000 cP), so as to be readily injected through a conventional small-bore hypodermic needle.

Accordingly, in one aspect of the invention the composition may include a drug combined with a high viscosity liquid carrier material (HVLCM) such as sucrose acetate isobutyrate (SAIB), together with any other suitable ingredients such as polymers, excipients, solvents, stabilizers, etc. In another aspect, the composition may comprise a combination of ingredients (e.g., biodegradable polymers, solvents, diluents, stabilizers, viscosity modifying agents, etc.) that allow the composition to exist in a liquid form prior to administration, but once the composition is introduced into the sinus, for example by injection (e.g., with a small bore needle) or as an aerosol or profoam, the composition can then form a gel, semi-solid or solid.

In certain compositions, the increase in viscosity of the composition after introduction into the sinus is due to removal of a solvent or viscosity-modifying substance from the composition, for example by diffusion and/or evaporation. The rate of solvent removal can be modulated and controlled, for example, by using a hydrophilic solvent that can be removed relatively quickly, or by using a hydrophobic solvent (e.g., benzyl benzoate) that will be removed from the composition relatively slowly. Combinations of such solvents may also be used. In other compositions of the invention, the increase in viscosity after introduction into the sinus can be the result of different mechanisms, for example, the composition can gel as a result of the increase in temperature from ambient conditions to body temperatures.

In other aspects of the invention, the compositions can be administered as a foamable composition, formulated with or without an added solvent. The composition that comprises drug, optional excipients and other ingredients, and a foaming agent (e.g., an aerosol propellant) is then administered into the sinus where decreased pressure and/or increased temperature causes the foaming agent to form gas bubbles in the composition. The resulting foam can then fill spaces inside the sinus such as a cavity, providing good, lasting contact of the composition with the interior sinus surfaces. Optionally the composition can further comprise excipients to help to stabilize the foam, either by hardening around the foam cells, or otherwise causing the foam to persist for sufficient time for delivery of the drug to occur.

In yet other aspects of the invention, prior to administration to the sinus the composition can be provided in the form of a liquid suspension of particles or microparticles comprising drug, or drug combined with carrier material, excipients and other optional ingredients.

The compositions of the invention may be formulated to degrade over time, desirably allowing modulated release of any drugs included therein. In this manner, all, or at least the bulk of the composition does not need to be retrieved from the sinus after drug delivery has been completed since the composition can be metabolized and/or cleared by processes that occur naturally in the subject. In a particular aspect of the invention, the composition or a substantial portion thereof is biodegradable or bioabsorbable. “Biodegradable” or “bioerodible,” used interchangeably herein, means that a subject material, composition or component thereof will degrade or erode in vivo to form smaller chemical species, wherein such degradation can result, for example, from enzymatic, chemical, and physical processes. “Bioabsorbable” means that a given material, composition or component thereof can be broken down and absorbed within a subject's body, for example, by a cell, tissue or the like.

Accordingly, in one primary embodiment the invention relates to the use of a carrier material for the manufacture of a drug delivery composition for delivering a drug to a subject, where the composition includes the carrier material combined with a drug of interest and is adapted specifically for administration directly into a sinus in the subject. The composition has no fixed shape prior to administration, and the carrier material serves to increase the viscosity or specific volume of the composition after it is introduced into the sinus, possibly imparting a suitable shape to the administered composition. The compositions manufactured in accordance with the invention can thus provide for safe and effective local and/or systemic intrasinus administration of drugs, and such administration can be accomplished with decreased levels of discomfort and invasiveness.

In one particular aspect, the invention relates to the use of HVLCM for the manufacture of a drug delivery composition for delivering a drug to a subject. The drug delivery composition thus comprises:

(a) one or more drug;

(b) the HVLCM; and optionally

(c) one or more excipient or additional ingredient, wherein the drug delivery composition is adapted to increase in viscosity or specific volume after introduction into the sinus.

In another particular aspect, the invention relates to the use of a carrier material that can form a gel, semi-solid, foam or high viscosity liquid after introduction into the sinus, where the material is used for the manufacture of a drug delivery composition adapted for delivery of drug to the sinus.

In another primary embodiment, the invention relates to a method for delivering a drug to a subject. The method entails administering a drug delivery composition directly to a sinus in the subject, where the composition includes a carrier material combined with the drug of interest. The drug is released from the composition to the sinus after administration. The initial composition has no fixed shape, and the carrier material serves to increase the viscosity or specific volume of the composition after it is introduced into the sinus. The methods of the invention can thus provide for safe and effective local and/or systemic intrasinus administration of drugs, and such administration can be accomplished with decreased levels of discomfort and invasiveness.

In one particular aspect, the method entails administering a drug delivery composition directly to a sinus in the subject, where the composition includes a HVLCM. The drug delivery composition thus comprises:

(a) one or more drug;

(b) the HVLCM; and optionally

(c) one or more excipient or additional ingredient, wherein the drug delivery composition is adapted to increase in viscosity or specific volume after introduction into the sinus.

The compositions of the present invention can be used to administer a drug or therapeutic agent to treat a disease or condition of the sinus or surrounding tissue (local or targeted administration), or may be administered to treat a disease or condition that is not limited to the sinus or surrounding tissue (systemic administration).

Use of the sinus as a repository for a drug delivery composition intended for systemic administration of a drug has several advantages. For example, administration into the sinus avoids the type of rapid and significant elimination or destruction of drug that occurs in the gastrointestinal tract during oral delivery methods. Instead, administration into the sinus provides enhanced residence time in the body (a decreased clearance time as compared to administration methods where the drug passes through or into the GI tract). In addition, administration into the sinus can provide a bolus of drug near a mucous membrane, which provides rapid introduction of the drug into the subject's circulatory system. As a result of these advantages, the bioavailability of the drug may be enhanced, and it may also be possible to decrease the dosage necessary to achieve a desired therapeutic result. Moreover, because the subject cannot access the interior of the sinus, the potential for tampering with the drug material after administration is reduced or eliminated, and patient compliance is improved.

An additional advantage provided by the instant compositions, particularly those incorporating a HVLCM and/or forming a gel, solid, or semisolid after administration, is that the compositions may be readily administered using routine procedures and common medical tools, devices and implements, e.g., those compositions that include a biocompatible solvent or diluent at least in part to temporarily provide a lower viscosity are perfectly well suited for administration using a small gauge needle. In this regard, a liquid of relatively low viscosity, e.g., between about 50 and about 2,000 cP can be administered through a needle having a gauge size ranging from about 18 to about 25 G. A slightly higher viscosity liquid, e.g., with a viscosity range of 2,000 to 10,000 cP, can be administered using a 12 to 18 G needle. A relatively low viscosity also promotes administration in conjunction with an aerosol propellant, which can allow the composition to be effectively coated on at least a portion of the internal surfaces of the sinus. After administration, the nature of the solvent or diluent allows it to rapidly leave the composition, with the result that the composition regains high viscosity and/or increases in volume to provide for increased retention within the sinus. Suitable solvents and diluents may be water-miscible or water-immiscible. Inclusion of optional polymer materials in the composition may further aid retention.

The invention is also advantageous in that the composition can serve as a platform for prolonged or sustained delivery of a drug or therapeutic agent to the tissues of the sinus and, the bulk of the composition can be biodegradable or otherwise cleared by natural body processes eliminating the need to retrieve the spent composition after the drug delivery event is finished. In addition, the invention possesses a number of advantages not found with pre-formed polymeric implants and stents. For example, standard pharmaceutically acceptable polymers can be included to increase the ability of the composition to fill a certain sinus space (e.g., cavity) or a portion thereof, and to then achieve and retain the shape necessary to remain in situ. Hydrophilic compounds can also be added to increase the muco-adhesive nature of the composition, providing the ability to avoid clearance by cilia, mucous or other fluid flow, and other natural transport and clearance mechanisms. In addition, because the compositions of the invention can be modified by including ingredients that alter the drug release profile characteristics, the invention is versatile, and can be used to administer a variety of drugs. Problems associated with systemic administration of drugs because of limited blood flow to the intrasinus membranes can be reduced or eliminated by the inclusion of certain vasoactive agents in the formulation that can increase blood flow to the membranes.

### Manufacture of the Compositions

The compositions of the present invention can be prepared by combining suitable pharmaceutical ingredients with the drug that is to be delivered to the sinus. Standard pharmaceutical methods are used to combine or otherwise mix the following ingredients.

If the composition is one that will form a gel or semisolid upon or after administration into the sinus, it may include one or more of a variety of gel bases such as carbomer, liquid paraffin, water, glycerol, propylene glycol, hyaluronic acid or sodium hyaluronate, or a combination thereof. Other types of suitable gel-forming materials include those that undergo gelling when the material exceeds a lower critical solution temperature (LCST), such as polyethylene oxide-polypropylene oxide block copolymers (PEO-PPO) (e.g., Poloxamer® or Pluronic® (BASF)), N-isopropylacrylamide (NIPA), copolymers of N-hydroxysuccinimidyl acrylate and acrylic acid, graft copolymers of polyacrylic acid (backbone) and PEO-PPO, graft copolymers of polyacrylic acid (backbone) and NIPA, chitosan backbone with PEO-PPO grafts, diblock copolymers of PEO and polylactic acid (PLA), triblocks of PEO-PLA-PEO, and copolymers of polycaprolactone and PEO. In general, as the composition increases in temperature from ambient temperatures to near body temperature, it gels, forming a drug delivery vehicle.

Another type of suitable gel-forming or semi-solid-forming material that may be included in the composition is a liquid crystalline material, such as solvated fatty acids of glycerol (e.g., glycerol monooleate). These materials change phase to a more highly viscous form upon injection and dilution with water. These materials can be combined with other suitable ingredients, as described in more detail below, to facilitate or increase injectability (e.g., oils, such as sesame oil), or to affect the drug release kinetics of the composition.

If the composition is of the type capable of undergoing polymer precipitation, the subject composition may include water-insoluble polymers that are dissolved in water-soluble solvents to make them injectable. After injection or other administration into the sinus, the solvent can diffuse away from the polymer which then coagulates or precipitates in the presence of the aqueous environment provided by the surrounding tissue in the sinus. Examples of suitable polymers that may be used in such compositions can include polylactides, lactide/glycolide copolymers, lactide/caprolactone copolymers, polyanhydrides, polyorthoesters, polyurethanes, and polycarbonates. Exemplary solvents that can be selected for use in these type of compositions can include N-methyl-2-pyrrolidone (NMP), 2-pyrrolidone, ethyl lactate, dimethylsulfoxide (DMSO), solketal, glycerol formal, propylene carbonate, ethyl acetate, and triacetin.

Still other alternative compositions can comprise a low molecular weight acryloyloxy-terminated oligomer or prepolymer (that is in liquid form) that can be combined with a cross-linking agent such as a peroxide just prior to administration. In this manner, the composition remains liquid during administration, and the resulting cross-linking reaction occurs in situ in the sinus, forming a gel or semi-solid bolus of hydrophobic polymer.

If the composition is of the type that will form a foam upon or after administration, it may include a foaming agent such as a foam stabilizer or a blowing agent (e.g., suitable foaming agents include aerosol propellants such as Dymel 134-A or dimethyether and gases such as nitrogen or carbon dioxide). Foam stabilizing agents such as biodegradable polymers or non-biodegradable polymers described below may be used to increase the rigidity and stability of the foam wall.

The characteristics of the composition can be readily varied by a wide choice of ingredients that can be added to provide for sustained or controlled release of the drug over a period of time. During this release period, the composition is not substantially cleared from the sinus. Following release of the desired amount of drug, some or all of the composition may degrade or otherwise be cleared from the sinus.

Exemplary drugs for use in the manufacture of the instant compositions include organic molecules, such as carbohydrates (including monosaccharides, oligosaccharides, and polysaccharides), steroids, nucleic acids (any form of DNA, including genes, cDNA, or RNA, or a fragment thereof), nucleotides, nucleosides, oligonucleotides (including antisense oligonucleotides), lipids, immunosuppressants, antioxidants, anesthetics, chemotherapeutic agents, steroids (including retinoids), antibiotics, antivirals, antifungals, antiproliferatives, anticoagulants, antiphotoaging agents, antimucosals, melanotropic peptides, nonsteroidal and steroidal anti-inflammatory compounds, antipsychotics, and radiation absorbers, including UV-absorbers, chemotherapeutic agents, anti-nausea medication, anti-infectives such as nitrofurazone, sodium propionate, antibiotics, including penicillin, tetracycline, oxytetracycline, chlorotetracycline, bacitracin, nystatin, streptomycin, neomycin, polymyxin, gramicidin, chloramphenicol, erythromycin, and azithromycin; sulfonamides, including sulfacetamide, sulfamethizole, sulfamethazine, sulfadiazine, sulfamerazine, and sulfisoxazole, and anti-virals including idoxuridine; antiallergenics such as antazoline, methapyritene, chlorpheniramine, pyrilamine prophenpyridamine, hydrocortisone, cortisone, mometasone, hydrocortisone acetate, dexamethasone, dexamethasone 21-phosphate, fluocinolone, triamcinolone, medrysone, prednisolone, prednisolone 21-sodium succinate, and prednisolone acetate; desensitizing agents such as grass and ragweed pollen antigens, tree and hay fever pollen antigens, house dust mite antigens and milk antigen; vaccines such as those for smallpox, yellow fever, distemper, hog cholera, chicken pox, antivenom, scarlet fever, diphtheria toxoid, tetanus toxoid, pigeon pox, whooping cough, influenza, rabies, mumps, measles, poliomyelitic, and Newcastle disease; decongestants such as phenylephrine, naphazoline, and tetrahydrazoline; miotics and anticholinesterases such as pilocarpine, esperine salicylate, carbachol, diisopropyl fluorophosphate, phospholine iodide, and demecarium bromide; parasympatholytics such as atropine sulfate, cyclopentolate, homatropine, scopolamine, tropicamide, eucatropine, and hydroxyamphetamine; sympathomimetics such as epinephrine; sedatives and hypnotics such as pentobarbital sodium, phenobarbital, secobarbital sodium, codeine, (a-bromoisovaleryl) urea, carbromal; CNS stimulants or depressants, including opioids, such as morphine, methadone, etorphine, levorphanol, fentanyl, sufentanil, [D-Ala(2)N-MePhe(4)Gly-ol(5)]enkephalin, (DAMGO), butorphanol, buprenorphine, naloxone, naltrexone, (Cys(2), Tyr(3), Orn(5), Pen(7)-amide (CTOP), diprenorphine, β-funaltrexamine, naloxonazine, nalorphine, pentazocine, nalbuphine, naloxone benzoylhydrazone, bremazocine, ethylketocyclazocine, U50,488, U69,593, spiradoline, nor-binaltorphimine, naltrindole, [d-Pen2,D-Pen5]enkephalin (DPDPE), [D-Ala²,Glu⁴]deltorphin, D-Ser-Leu-enkephalin-Thr (DSLET), Met-enkephalin, Leu-enkephalin, β-endorphin, dynorphin A, dynorphin B, α-neoendorphin, heroin, hydromorphone, oxymorphone, levallorphan, codeine, hydrocodone, oxycodone, and nalmefene; psychic energizers such as 3-(2-aminopropyl) indole acetate and 3-(2-aminobutyl) indole acetate; tranquilizers such as reserpine, chlorpromayline, and thiopropazate; hormones, including androgenic steroids such as methyl-testosterone and fluorymesterone, estrogens such as estrone, 17-β-estradiol, ethinyl estradiol, and diethyl stilbestrol, and progestational agents such as progesterone, megestrol, melengestrol, chlormadinone, ethisterone, norethynodrel, 19-norprogesterone, norethindrone, medroxyprogesterone and 17-β-hydroxy-progesterone; humoral agents such as the prostaglandins, for example PGE₁, PGE₂ and PGF₂; antipyretics such as aspirin, sodium salicylate, and salicylamide; antispasmodics such as atropine, methantheline, papaverine, and methscopolamine bromide; antimalarials such as the 4-aminoquinolines, 8-aminoquinolines, chloroquine, and pyrimethamine, antihistamines such as diphenhydramine, dimenhydrinate, tripelennamine, perphenazine, and chlorphenazine; cardioactive agents such as dibenzhydroflume thiazide, flumethiazide, chlorothiazide, and aminotrate; nutritional agents such as vitamins, including vitamin C and vitamin E, natural and synthetic bioactive peptides and proteins, including nucleoproteins, mucoproteins, lipoproteins, glycoproteins, small molecules linked to proteins, growth factors, cell adhesion factors, cytokines, and biological response modifiers.

The drug is included in the composition in an amount sufficient to deliver to the subject an effective amount to achieve a desired effect. The amount of drug incorporated into the composition depends, inter alia, upon the desired release profile, the concentration of drug required for a biological effect, and the desired period of release of the drug, all of which variables are readily determined by one of ordinary skill in the pharmaceutical arts.

The concentration or amount of drug present in the composition will also depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens may be adjusted over time according to the individual need and the professional judgment of the person administering or supervising the administration of the compositions, and that any express concentration ranges set forth herein are exemplary only and are not intended to limit the scope or practice of the claimed compositions. The composition may be administered in one dosage, or may be divided into a number of smaller doses to be administered at varying intervals of time and/or to different parts of the sinus.

Classes of drugs of particular interest for delivery using the methods disclosed herein are those used to treat sinusitis and other disease conditions of the nasal sinus. These include steroidal and non-steroidal anti-inflammatory agents (e.g., mometasone), anti-infectives, including antibiotics and antivirals, antifungal agents, and the like.

As discussed above, various solvents or diluents can be included in the composition for the purpose of lowering the viscosity temporarily, or for the purpose of dissolving the drug and other components such as polymers, rheology modifiers, stabilizers and other additives. Solvents useful in the practice of the present invention include, but are not limited to, alcohols, organic acids and their derivatives, esters of organic acids, and compounds possessing an alcohol and an organic acid residue e.g., ethyl lactate (EL) or triacetine, DMSO, propylene carbonate, NMP, ethyl alcohol, benzyl alcohol, glycofurol, Miglyol 810, and benzyl benzoate.

The solvents that are used in the manufacture of the instant compositions are desirably biocompatible and do not cause significant tissue irritation or necrosis in the sinus, unless irritation or necrosis is the desired effect.

In certain compositions, the solvent may be at least water soluble so that it will diffuse quickly into bodily fluids or other aqueous environment in the sinus, causing the remaining composition to coagulate or solidify. In other compositions, the solvent is not water soluble and does not quickly diffuse away. Particular examples of suitable solvents include alcohols, such as ethanol and miglyol; organic acids and their derivatives, such as oleic acid; and organic acid esters, such as ethyl lactate, methyl acetate, and triacetin; as well as other common organic solvents such as propylene carbonate, glycofurol, NMP, 2-pyrrolidone, propylene glycol, acetone, methyl ethyl ketone, benzyl alcohol, dimethylformamide, dimethylsulfoxide, tetrahydrofuran, caprolactam, decylmethylsulfoxide, and 1-dodecylazacycloheptan-2-one.

When the composition includes a HVLCM such as SAIB as the carrier material, preferred solvents include ethanol, dimethylsulfoxide, ethyl lactate, ethyl acetate, benzyl alcohol, triacetin, N-methylpyrrolidone, propylene carbonate, glycofurol, Miglyol 810 and benzyl benzoate. SAIB is not miscible with certain materials such as glycerol, corn oil, peanut oil, 1,2-propanediol, polyethylene glycol (PEG200), super refined sesame oil, and super refined peanut oil. Accordingly, the latter group of solvents are not preferred for use with SAIB, although they can be present as co-solvents or used to provide emulsions.

When a solvent is included in the composition, the solvent is typically added in an amount in the range from about 2 percent to about 55 percent by weight, relative to the total weight of the composition. Preferably, the solvent is present in the composition in an amount ranging from about 5 percent to about 50 percent by weight. Another preferred range is from about 10 percent to 30 percent by weight.

Other ingredients may be included to modify the properties of the compositions in various ways. These additional (and optional) ingredients include polymers that can be used to stiffen or increase the structural stability of gels, semisolids, or foams, as well as polymers that can be used to adjust the release profile characteristics of the drug. Because the composition is administered initially in a liquid form, it can subsequently take the shape of the sinus, such as the shape of a sinus cavity or portion thereof. As the composition increases in viscosity, solidifies, or foams, polymeric additives help to stiffen it, causing it to retain its good surface contact with the sinus tissue, providing a high surface area over which release and transfer of the drug can occur. With foamed materials, a foaming agent is used to disperse the liquid, increasing the specific volume of the composition and expanding it to fill some or all of the sinus spaces, again providing good surface area contact with the sinus walls and tissues. In regard to foam compositions, this large surface area is obtained with low mass since a significant portion of the foam is gaseous. Polymeric additives can also be added to stiffen the foam, allowing it to retain a low-density structure, even after the foaming agent has ceased to supply additional gas to the foam. These polymeric materials include PLG, PLA, and other biodegradable and non-biodegradable polymers.

Additional additives for use in the manufacture of the instant include one or more bioadhesive materials, although this is not strictly necessary for the compositions to be suitable in the methods of the invention. Accordingly, the composition can include hydrophilic compounds to provide mucoadhesion to the sinus walls. These include polymers that, upon moistening, swell and achieve sufficient tack or otherwise become adhesive. Examples of mucoadhesive polymers that may be employed in the compositions of the invention include homopolymers of acrylic acid monomers such as polyacrylic acid and any of its pharmaceutically acceptable salts; copolymers of acrylic acid and methacrylic acid, styrene, or vinyl ethers; vinyl polymers such as polyhydroxyethyl acrylate, polyhydroxyethyl methacrylate, polyvinyl alcohol, and polyvinyl pyrrolidone; cellulosic derivatives such as methyl cellulose, ethyl cellulose, hydroxyethyl cellulose, hydroxypropyl cellulose, hydroxypropylmethyl cellulose, and carboxymethyl cellulose; polysaccharides such as alginic acid, sodium alginate, and tragacanth gum; collagen; gelatin; and any combination thereof.

As disclosed herein, the characteristics of the compositions can readily be varied by the inclusion of various optional ingredients. These additional ingredients can include variety of different types of substances, examples of which are described below. The ingredients can be present in any amount sufficient to impart the desired properties to the composition, e.g., viscosity, retention, degradation, injectability, biocompatibility, and drug release, distribution and elimination. The amount of such additional ingredients included will in general be a function of the nature of the ingredient and the effect to be achieved, and can be easily determined by one of skill in the art in light of the disclosure provided by the instant specification. Accordingly, such ingredients may be present in an amount ranging from about 0.1 percent to about 90 percent by weight relative to the total weight of the composition and, more typically, are present in the composition in an amount ranging from about 1, 2, or 5 percent to about 35 to 30 percent by weight. Certain ingredients, such as buffers, are typically only present in small amounts in the composition.

The following categories are nonlimiting examples of classes of ingredients that can be employed in the composition. Given the disclosure herein and the objects to be achieved, one of skill in the art will easily know how to select other additives to achieve a desired purpose.

(HVLCMs) In certain preferred compositions, the carrier material comprises a HVLCM (e.g., SAIB) that is matched with a biocompatible solvent to reduce the viscosity of the initial composition to ease administration into the sinus. Exemplary compositions comprising SAIB are set forth in the examples below. In general, the compositions may include any non-polymeric, non-water soluble biocompatible liquid material having a viscosity of at least 5,000 cP at 37° C., particularly those that do not crystallize neat under physiological conditions. HVLCMs used herein may be carbohydrate-based, and may include one or more cyclic carbohydrates chemically combined with one or more carboxylic acids, such as Sucrose Acetate Isobutyrate (SAIB). HVLCMs also include nonpolymeric esters or mixed esters of one or more carboxylic acids, having a viscosity of at least 5,000 cP at 37° C., that do not crystallize neat under ambient or physiological conditions. The ester may, for example comprise from about 2 to about 20 hydroxy acid moieties. Various HVLCMs suitable for use with the present drug-delivery system are described in U.S. Pat. Nos. 5,747,058; 5,968,542; and 6,413,536, as well as in U.S. Ser. Nos. 09/699,002, filed Oct. 26, 2000 and 10/316,441, filed Dec. 10, 2002, each incorporated in their entirety by reference herein.

(Biodegradable Polymers) Another specific category of ingredients that can be included in the instant compositions are biodegradable polymers and oligomers. The polymers can be used to alter the release profile of the drug to be delivered, to add integrity to the composition, or to otherwise modify the properties of the composition. Non-limiting examples of suitable biodegradable polymers and oligomers include: poly(lactide), poly(lactide-co-glycolide), poly(glycolide), poly(caprolactone), polyamides, polyanhydrides, polyamino acids, polyorthoesters, polycyanoacrylates, poly(phosphazines), poly(phosphoesters), polyesteramides, polydioxanones, polyacetals, polyketals, polycarbonates, polyorthocarbonates, degradable polyurethanes, polyhydroxybuty-ates, polyhydroxyvalerates, polyalkylene oxalates, polyalkylene succinates, poly(malic acid), chitin, chitosan, and copolymers, terpolymers, oxidized cellulose, or combinations or mixtures of the above materials.

Examples of poly(alpha-hydroxy acid)s include poly(glycolic acid), poly(DL-lactic acid) and poly(L-lactic acid), and their copolymers. Examples of polylactones include poly(epsilon-caprolactone), poly(delta-valerolactone) and poly(gamma-butyrolactone).

(Non-biodegradable Polymers) Yet another particular class of ingredients suitable for use with the present compositions includes non-biodegradable polymers. Non-limiting examples of non-biodegradable polymers which can be used to manufacture the present compositions include: polyacrylates, ethylene-vinyl acetate polymers, cellulose and cellulose derivatives such as HPMC, acyl substituted cellulose acetates and derivatives thereof, non-erodible polyurethanes, polystyrenes, polyvinyl chloride, polyvinyl fluoride, poly(vinyl imidazole), chlorosulphonated polyolefins, and polyethylene oxide.

Preferred non-biodegradable polymers include polyethylene, polyvinyl pyrrolidone, ethylene vinylacetate, polyethylene glycol, cellulose acetate butyrate (“CAB”) and cellulose acetate propionate (“CAP”), acrylate polymers, such as polyacrylic acid, and hyaluronic acid.

(Thickeners) A still further class of ingredients that can be used in the present compositions includes natural and synthetic oils and fats. Oils derived from animals or from plant seeds of nuts typically include glycerides of the fatty acids, chiefly oleic, palmitic, stearic, and linolenic. As a general rule, the more hydrogen the molecule contains, the thicker the oil becomes. Other suitable thickeners include natural gums, carbohydrates, and starches, such as gum arabic, gum tracaganth, guar gum, karaya gum, agar, carrageenan, and other polysaccharides. These thickeners can contribute to the stabilization of foams in foaming compositions of the invention.

Non-limiting examples of suitable natural and synthetic oils include vegetable oil, peanut oil, medium chain triglycerides, soybean oil, almond oil, olive oil, sesame oil, peanut oil, fennel oil, camellia oil, corn oil, castor oil, cotton seed oil, and soybean oil, either crude or refined, and medium chain fatty acid triglycerides.

Fats are typically glyceryl esters of higher fatty acids such as stearic and palmitic. Such esters and their mixtures are solids at room temperatures and exhibit crystalline structure. Lard and tallow are examples. In general, oils and fats increase the hydrophobicity of the composition, slowing degradation and water uptake.

(Carbohydrates and Carbohydrate Derivatives) Another class of ingredients useful herein includes carbohydrates and carbohydrate derivatives. Non-limiting examples of such materials include monosaccharides (simple sugars such as fructose and its isomer glucose (dextrose); disaccharides such as sucrose, maltose, cellobiose, and lactose; and polysaccharides.

Other suitable ingredients include anti-oxidants and preservatives, such as BHA, BHT, or alpha-tocopherol and the like. These materials are typically added to increase the storage stability of the composition and provide other desirable properties that may be unrelated to the release kinetics of the composition. These include surfactants, such as SDS, TWEEN, TRIS, and polyvinyl alcohol; minerals, such as zinc, magnesium or calcium salts, and the like.

### Methods for Using the Compositions

The compositions of the present invention can be administered in the form of a liquid that is sufficiently flowable such that it can be introduced into a sinus through a small bore hypodermic needle. As described above, this is generally a needle of a size ranging from 18 to 25 gauge for a formulation of viscosity between about 50 and about 1,000 cP, although larger bore needles (e.g., 12 to 18 gauge) can be used with higher viscosity formulations. The needle bore can be brought into communication with the interior of the sinus either by piercing the sinus wall or, in the case of the maxillary nasal sinus, through the arafural ostium or openings made through the anterior, posterior or inferior walls. Depending in part on the nature of the composition (e.g., whether in the form of a HVLCM, a foam, a gel, etc.), the composition once inside the sinus will form a drug release bolus or depot, or a foam that takes the shape of at least a portion of the sinus, or can be sprayed through the hypodermic onto the surfaces of the sinus, where it forms a high viscosity film.

Once administered into the sinus, the composition will deliver drug to the sinus for either local or systemic delivery thereof. Because the flowable or foamable nature of the composition allows it to conform to the anatomy of the sinus, or alternatively to be coated across at least a portion of the surface of the sinus tissue and walls, excellent contact with the sinus tissue is assured, leading to very effective local distribution of the composition through the targeted sinus.

While any drug may be delivered into the sinus using the compositions and methods of this invention, the instant methods are particularly well adapted for the intrasinus administration of drugs that are suitable for treating disease conditions and/or injuries occurring in the sinus itself. These include sinusitis, also referred to as rhinosinusitis, as well as a broad spectrum of inflammatory and infectious diseases concurrently affecting the nose and paranasal sinuses including bacterial, viral, and fungal infections. Any and all of these disorders may be caused by systemic host factors such as allergies, immunodeficiency, genetic/congenital, mucociliary dysfunction, endocrine or neuromechanisms including local host factors such as anatomic or neoplastic abnormalities and including environmental factors such as microorganisms, viral, bacterial and fungal as well as noxious chemicals, pollutants, smoke, medications, trauma and surgery. Antibacterials, antivirals, antifungals, antiinflammatories (steroidal and non-steroidal) immunomodulators and decongestants can all be delivered by the methods and compositions of the invention.

In addition, the compositions and methods of the invention can be used to deliver anesthetic compounds during surgery or subsequent healing, as well as antiseptic to speed healing of injuries to the sinus (e.g., of injuries caused by inhalation of caustic or other damaging compounds).

For many of these treatments, the release profile of drug over the release period is preferably approximately steady over time, sufficient to provide a therapeutic dose over the release period, and preferably shows a decreased burst effect when compared to a standard composition containing the same drug. The time to 90% release of drug may be controlled by varying the composition components, and may be as little as 4 hours, 6 hours, 8 hours, 10, hours, 12 hours, 16 hours or 2 weeks, or up to about 4 weeks or one or more months.

The rate of drug release from the composition may be varied depending on the drug used and dosage required. Release rates may be different in different sinuses and in different parts of any particular sinus, and may be different for a composition that is deposited as a depot by injection and for a composition that is sprayed over the inner surfaces of the sinus. Release rates may vary depending on the drug, and can be for example, from about 0.01 to 500 μg/hr, from 0.5 to 250 μg/hr, 0.75 to 100 μg/hr, 1.0 to 100 μg/hr, 2.0 to 100 μg/hr, 5 to 100 μg/hr, 10 to 100 μg/hr, 10 to 80 μg/hr, 20 to 50 μg/hr, or about 20 to 40 μg/hr.

Below are examples of specific embodiments for carrying out the methods of the present invention. The examples are offered for illustrative purposes only, and are not intended to limit the scope of the present invention in any way.

### EXAMPLES

### Example 1

SAIB and NMP are combined in a weight ratio of 92:8. Mometasone is added to this solution in an amount of 7.5 mg/ml, and 100 microliters of the resulting composition is injected through a small bore needle into the maxillary sinus of a rabbit, where it forms a small, high viscosity liquid bolus in the lower portion of the sinus cavity. Mometasone delivery over a period of at least 30 days results.

### Example 2

SAIB, DYMEL 134-a (a foaming agent), and a 75:25 poly(dl-lactide-co-glycolide) having an inherent viscosity of 0.20 dL/g at 30° C. are mixed at a weight ratio of 48:50:2 in a glass aerosol vessel. The resulting composition will foam to a low density foam after administration and maintain this shape in an aqueous system (water) at 37° C. for more than 2 weeks.

### Example 3

A solution of POLOXAMER 407 (BASF) and water is prepared by mixing the two at a 35:65 weight ratio. Finely ground mometasone (10 mg/ml) is added to the solution to form a suspension, which is injected through a small bore needle into the maxillary sinus of a rabbit. After injection, viscosity will increase, and mometasone will be released over a period of at least 1 week.

### Example 4

A moderately viscous mixture of glycerol monooleate and 15 wt % water is prepared by mixing. Finely ground mometasone is added to form a suspension (5 mg/ml), which, when injected through a small bore needle into the maxillary sinus of a subject, will provide detectable levels of mometasone for at least 2 weeks.

### Example 5

A viscous solution of 5 wt % hyaluronic acid in water is prepared by mixing. Finely ground mometasone is added, forming a suspension (7.5 mg/ml). The suspension is injected through a small-bore needle into the maxillary sinus.

### Example 6

SAIB and benzyl benzoate are combined at a 75:25 ratio. To this is added 2.5 mg/ml of mometasone furoate solution. 0.5 mL of this composition is injected into the maxillary sinus where it provides for extended release of drug.

### Example 7

A mixture of mometasone furoate and poly(lactide) is prepared by mixing 1 part of drug with 3 parts of polymer in acetone. The acetone is evaporated, and the resulting solid is ground to a fine particle. The particle of drug/polymer mix in suspended in a solution of SAIB, ethanol, such that the final mixture is 72% SAIB, 24% ethanol, 3% polymer, and 1% drug.

### Example 8

An aerosol solution is prepared with a SAIB, NMP mixture, where the ratio of SAIB:NMP is 4 parts to 1. This solution is mixed with an equal part of DYMEL 134-A. The resulting solution can be sprayed into a sinus where it will form a thin adherent film.

What is claimed is:

1. Use of a carrier material for the manufacture of a drug delivery composition for delivering a drug to a subject, wherein the composition comprises the carrier material combined with the drug, the composition is for insertion directly into a sinus in the subject and the composition has no fixed shape, and further wherein the carrier material serves to increase the viscosity or specific volume of the composition after introduction of the composition into the sinus.

2. The use of claim 1, wherein the composition forms a gel, semi-solid, foam or high viscosity liquid after introduction into the sinus.

3. The use of claim 1 or 2, wherein the composition forms a drug delivery depot in situ in the sinus.

4. The use of any one of claims 1-3, wherein the increase of viscosity or specific volume is suitable to fill at least a portion of a targeted sinus space.

5. The use of any one of claims 1-4, wherein the composition is at least partly biodegradable.

6. The use of any one of the preceding claims, wherein the composition is adapted to at least partially clear from the sinus after delivering an effective amount of the drug.

7. The use of any one of the preceding claims, wherein the carrier material provides for controlled release of the drug to the sinus.

8. The use of any one of the preceding claims, wherein the carrier material comprises a high viscosity liquid carrier material (HVLCM).

9. The use of claim 8, wherein the HVLCM comprises a sucrose acetate isobutyrate.

10. The use of any one of the preceding claims, wherein the composition further comprises a solvent capable of diffusing away from the composition after administration, thereby increasing the viscosity of the remaining composition.

11. The use of any one of the preceding claims, wherein the composition further comprises a mucoadhesive agent.

12. The use of any one of the preceding claims, wherein the drug comprises an antifungal agent, anti-inflammatory agent, anti-infective agent, or any combination thereof.

13. The use of claim 12, wherein the drug comprises mometasone.

14. The use of any one of the preceding claims, wherein the composition is administered to the sinus by spraying the composition onto a surface of the sinus.

15. The use of any one of claims 1-13, wherein the composition is administered to the sinus by injection.

16. The use of any one of claims 1-13, wherein the composition is administered to the sinus through an opening in a sinus cavity.

17. A method for delivering a drug to a subject, said method comprising administering a drug delivery composition directly to a sinus in the subject, wherein the composition comprises a carrier material combined with the drug and has no fixed shape, and further wherein the drug is released from the composition to the sinus and the carrier material serves to increase the viscosity or specific volume of the composition after it is administered to the sinus.

18. The method of claim 17, wherein the composition forms a gel, semi-solid, foam or high viscosity liquid after introduction into the sinus.

19. The method of claim 17 or 18, wherein the composition forms a drug delivery depot in situ in the sinus.

20. The method of any one of claims 17-19, wherein the increase of viscosity or specific volume is suitable to fill at least a portion of a targeted sinus space.

21. The method of any one of claims 17-20, wherein the composition is at least partly biodegradable.

22. The method of any one of claims 17-21, wherein the composition is adapted to at least partially clear from the sinus after delivering an effective amount of the drug to the sinus.

23. The method of any one of claims 17-22, wherein the carrier material provides for controlled release of the drug to the sinus.

24. The method of any one of claims 17-23, wherein the carrier material comprises a high viscosity liquid carrier material (HVLCM).

25. The method of claim 24, wherein the HVLCM comprises a sucrose acetate isobutyrate.

26. The method of any one of claims 17-25, wherein the composition further comprises a solvent capable of diffusing away from the composition after administration, thereby increasing the viscosity of the remaining composition.

27. The method of any one of claims 17-26, wherein the composition further comprises a mucoadhesive agent.

28. The method of any one of claims 17-27, wherein the drug comprises an antifungal agent, anti-inflammatory agent, anti-infective agent, or any combination thereof.

29. The method of claim 28, wherein the drug comprises mometasone.

30. The method of any one of claims 17-29, wherein the composition is administered to the sinus by spraying the composition onto a surface of the sinus.

31. The method of any one of claims 17-29, wherein the composition is administered to the sinus by injection.

32. The method of any one of claims 17-29, wherein the composition is administered to the sinus through an opening in a sinus cavity
